(19) United States
(12) Patent Application Publication
(10) Pub. No.: US 2026/0174459 A1
(43) Pub. Date: Jun. 25, 2026

(54) SURGICAL DEVICE HAVING MULTIPLE MODES OF TISSUE REMOVAL

(71) Applicant: M. I. MILLINGATOR, INC., Hanover, NH (US)

(72) Inventors: KORNEL HORVATH, Budaors (HU); ZSOLT SZARAZ, Budapest (HU); BALAZS LESKO, Budapest (HU); GABOR VARADI, Budapest (HU)

(21) Appl. No.: 19/126,001

(22) PCT Filed: Nov. 2, 2023
(86) PCT No.: PCT/US2023/078531

§ 371 Date: Apr. 30, 2025

Related U.S. Application Data
(60) Provisional application No. 63/382,045, filed on Nov. 2, 2022.

(51) Int. Cl. A61B 17/32; A61B 17/16; A61B 17/3207

(52) U.S. Cl. CPC A61B 17/320016; A61B 17/1617; A61B 17/32002; A61B 17/320783

(57) ABSTRACT

A multiple mode cutting head is described having a closed configuration in which no cutting edge is presented. The multiple mode cutting head also has an off central axis inner cutting head with two radially separated scalloped cutting edges. An opening in an outer head has two straight cutting edges positioned for interaction with the scalloped edges during rotation of the inner cutting head.

### CROSS REFERENCE TO RELATED APPLICATIONS

This application claims the benefit of U.S. Provisional Patent Application No. 63/382,045, filed Nov. 2, 2022, the entire disclosure of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure relates to a motor-driven rotating tubular cutter surgical instrument having multiple different modes of operation to produce different methods for resecting tissue from the interior of a patient's body.

### BACKGROUND

A number of rotating cutting surgical instruments are available and in broad use. Despite the widespread availability of such instruments, a number of drawbacks in performance and various modalities suited to enhanced surgical control remain. Moreover, many existing devices present cutting surfaces and require the use of delivery sheaths of additional covers to be positioned over exposed cutting surfaces when being moved into and out of use in the surgical field. For at least these reasons, improved rotating surgical instruments are needed to address these and other drawbacks.

### SUMMARY OF THE DISCLOSURE

Described herein are embodiments of a surgical cutting head having a proximal end having a handle, and a distal end having a cutting head with an outer cutting head having an opening and two straight cutting edges and an inner cutting head positioned for eccentric rotation relative to the opening and in cutting relation to the two straight cutting edges. In another aspect, the surgical cutting head may also include a motor control system adapted and configured to control the direction and speed of rotation of the inner cutting head. In yet a further aspect, the surgical cutting head and motor control system may be operated in a manual mode, a semiautonomous mode or an autonomous mode. Still further, the cutting head has a closed mode where no cutting surface is exposed or only a smooth exterior surface of the cutting head is presented. In some embodiments, the surgical cutting head also includes a push button, a selector switch, a footswitch or a voice activated control system adapted and configured to automatically transition the cutting head into the closed mode.

In still further implementations, the surgical cutting head also includes one or more sensors coupled to a portion of the cutting head and a computer implemented tissue characterization and mode selection tissue cutting control system in electronic communication with the one or more sensors wherein based at least in part on a signal from the one or more sensors the computer implemented cutting control system recommends or automatically adjusts the motor control system between the manual mode, the semi-autonomous mode and the autonomous mode. Still other embodiments provide for the computer implemented tissue characterization and mode selection tissue cutting control system is configured to automatically transition the cutting head to the closed mode based on a determination of signals received from the one or more sensors.

In still additional alternatives and variations, there is also provided a method of removing bone, ligament, muscle, soft tissue, calcified tissue, scarred tissue, tumor or other targeted tissue using any of the devices and systems described herein.

In still further implementations, a method of removing bone, ligament, muscle, soft tissue, calcified tissue, scarred tissue, tumor, or other targeted issue is provided. The method comprises advancing a device comprising a cutting head with an outer cutting head having an opening and two straight cutting edges and an inner cutting head positioned for eccentric rotation relative to the opening and in cutting relation to the two straight cutting edges to a target tissue; selecting one or more cutting parameters based on properties of the target tissue; and rotating the inner cutting head relative to the outer cutting head, thereby removing tissue from the target tissue.

In further implementations, the method comprises selecting a mode of operation of the cutting device. In still further implementations, selecting a mode of operation comprises selecting between manual control, semi-autonomous control or autonomous control. In further implementations, the method comprises engaging various portions of a target tissue site with the cutting head to aid in selection of an appropriate cutting mode for the various portions of the target tissue site. The method can comprise modifying the mode of operation or mode of cutting based on feedback from sensors on the cutting head. In further implementations, rotating the inner cutting head relative to the outer cutting head comprises rotating the inner cutting head back and forth about 2-5 times at an RPM of about 2000-4000 RPM, which can comprise cutting through soft tissue. In further implementations, rotating the inner cutting head relative to the outer cutting head comprises rotating the inner cutting in one direction at an RPM of about 4000-8000 RPM, which can comprise cutting through cartilage and/or bone.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A, 1B and 1C are perspective views of an exemplary embodiment of a rotating surgical device. FIG. 1A illustrates the cutting head at the distal end of a device in a closed configuration in which no cutting surface is exteriorly presented. FIG. 1B illustrates the cutting head of FIG. 1A with the inner cutting head half opened exposing one scalloped cutting edge and one straight edge of the outer head. FIG. 1C is a view of the cutting head FIG. 1A with the inner cutting head fully open exposing both scalloped cutting edges and both straight cutting edges of the outer head (only one visible in this view).

FIGS. 2A and 2B are perspective views of the outer cutting head and inner cutting head.

FIGS. 3A and 3B are perspective and cross section views of the primary and secondary cutting edges with arrows indicating the direction of rotation and resulting variation in cutting surface engagement.

FIGS. 4A-4D are cross section views through the cutting head showing the interrelation of the cutting edges and resecting line of the outer cutting head and inner cutting head in a closed condition (FIG. 4A), a mid-opened condition (FIG. 4B), a fully opened condition (FIG. 4C) and fully open with indication of the resecting line in relation to the outer head (FIG. 4D).

FIGS. 5A-5C illustrate a perspective view of a prototype of an embodiment of a cutting head. FIG. 5A illustrates the cutting head at the distal end of a device in a closed configuration in which no cutting surface is exteriorly presented. FIG. 5B illustrates the cutting head of FIG. 5A with the inner cutting head half opened exposing one scalloped cutting edge and one straight edge of the outer head. FIG. 5C is a view of the cutting head FIG. 5A with the inner cutting head fully open exposing both scalloped cutting edges and both straight cutting edges of the outer head (only one visible in this view).

FIG. 6 is a first perspective view of a resected portion of a cadaver limb showing the effective removal of cartilage, ligament, bone and soft tissue via operation of the prototype cutting head of FIGS. 5A-5C.

FIG. 7 is a second perspective view of the resected portion of a cadaver limb of FIG. 6 showing the effective removal of cartilage, ligament, bone and soft tissue via operation of the prototype cutting head of FIGS. 5A-5C.

FIG. 8 is a flow chart detailing a number of different operating modes for the cutting head surgical system.

### DETAILED DESCRIPTION

The various embodiments of the inventive multi-mode cutting head may be advantageously applied to a wide range of surgical probes utilized in various types of surgeries, including but not limited to gynecology procedures such a myomectomies and polypectomies, ENT procedures, arthroscopies, spine surgeries, tumor resection procedures as well as general surgical removal, shaving or shaping procedures involving healthy, scarred, diseased bone, ligament, tendon, muscle, and soft tissue.

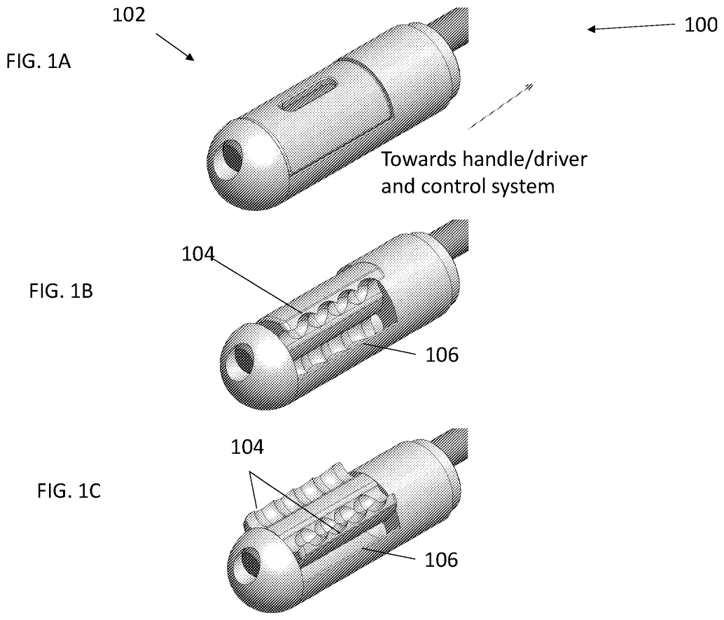

FIGS. 1A, 1B and 1C are perspective views of an exemplary embodiment of a rotating surgical device 100. FIG. 1A illustrates the cutting head 102 at the distal end of a device in a closed configuration in which no cutting surface is exteriorly presented. FIG. 1B illustrates the cutting head of FIG. 1A with the inner cutting head half opened exposing one scalloped cutting edge 104 and one straight edge 106 of the outer head. FIG. 1C is a view of the cutting head FIG. 1A with the inner cutting head fully open exposing both scalloped cutting edges 104 and both straight cutting edges 106 of the outer head (only one visible in this view).

In one aspect, the cutting head may remain in closed state (FIG.1A) as a base state for movement along an access or delivery scope or within or through the surgical site. Additionally, a user is able to rotate the inner cutting head into closed state, when desired. A sensor on a handle coupled to the cutting head along with a trigger signal generator part on the rotating head element. In one aspect, the sensor may be coupled to a scalloped inner head proximal side, which connects to the handle. In one aspect, the insertion and removal of the cutting head through a surgical access scope will be in closed state. In one embodiment, there is a button to be pressed on the handle before the insertion or removal process. A head open/head closed indicator may be used to warn the user of the actual state of the cutting head in relation to a desire head movement action.

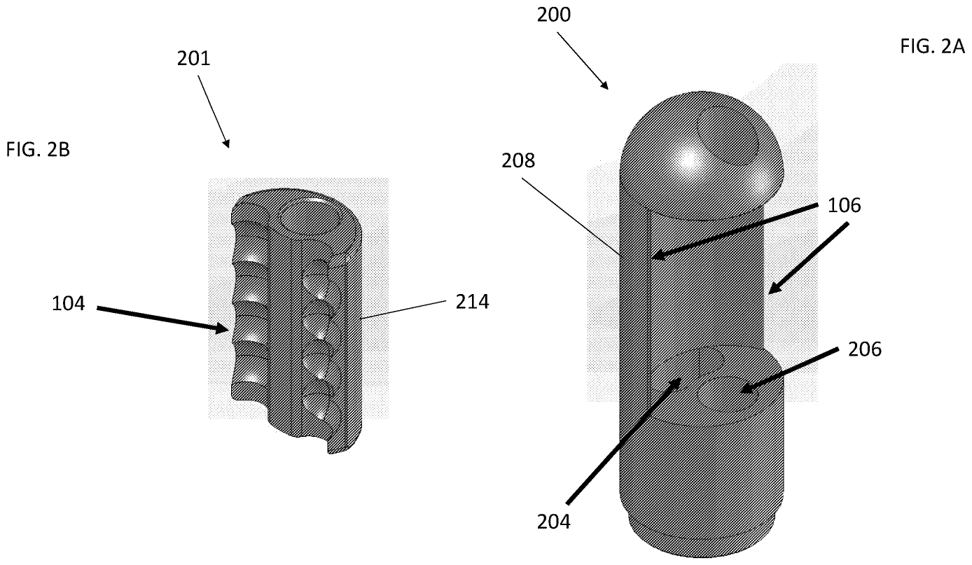

FIGS. 2A and 2B are perspective views of the outer cutting head 200 and inner cutting head 201, respectively. FIG. 2A shows the outer cutting head. The outer cutting head comprises a smooth portion 208. Also shown are the straight cutting edges 106. The outer cutting head also comprises a vacuum channel 204 and a drive shaft channel 206.

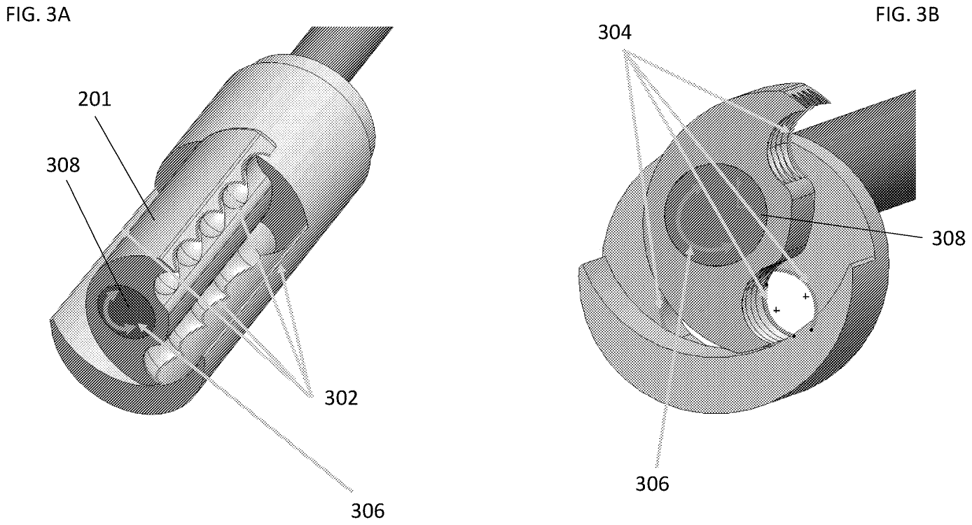

Moving now to FIG. 2B, the inner cutting head is shown. The inner cutting head comprises scalloped cutting edges 104. The inner cutting head comprises primary cutting edges and secondary cutting edges, described in more detail in FIGS. 3A and 3B. The inner cutting head also comprises a drive shaft channel 212. On an opposite side from the cutting surfaces is a smooth surface 214

Advantageously, during rotation, the cutting edges of the inner cutting head 201 also pass by the opening of the outer head 200 providing a meeting of the curved cutting blade on the rotating inner sleeve to the flat edge opening in the fixed outer sleeve.

FIGS. 3A and 3B are perspective and cross section views of the primary 302 and secondary 304 cutting edges with arrows 304 indicating the direction of rotation and resulting variation in cutting surface engagement. In some embodiments, the drive shaft 308 and inner cutting head 201 are configured to spin back and forth in the direction indicated by the arrow 306.

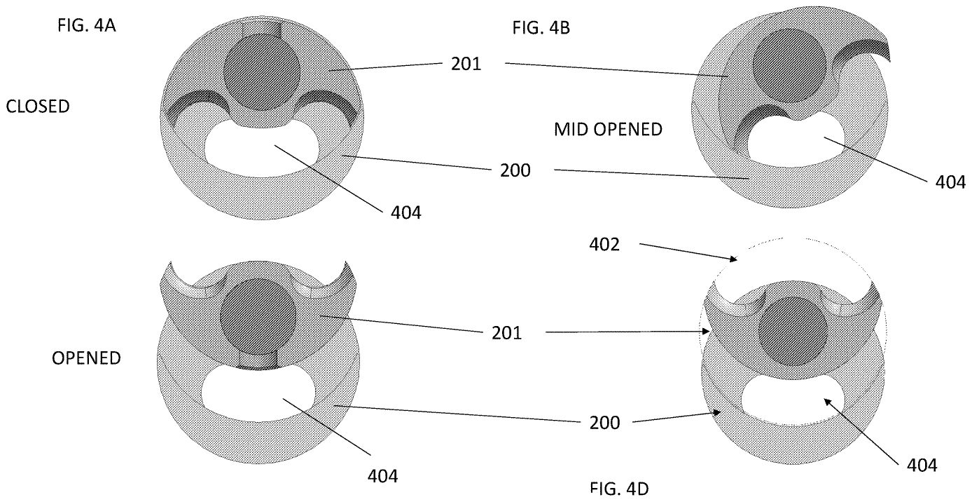

FIGS. 4A-4D are cross section views through the cutting head showing the interrelation of the cutting edges and resecting line 402 of the outer cutting head 200 and inner cutting head 201 in a closed condition (FIG. 4A), a mid-opened condition (FIG. 4B), a fully opened condition (FIG. 4C) and fully open with indication of the resecting line in relation to the outer head (FIG. 4D). The vacuum channel 404 is also visible in FIGS. 4A-4D.

As best seen in the various views of FIGS. 3A-4D , the axis of rotation of the inner cutting head is off axis of the central longitudinal axis of the overall cutting head. This stands in contrast to many conventional cutting devices where the axis of rotation of the cutting head is co-axial to the overall longitudinal axis or where all cutting heads in a multiple cutting head system share a common rotational axis. One benefit of the of central axis rotation of the inner cutting head is the resecting line illustrated in FIG. 4D.

In one aspect, a user may choose an automatic mode or a manual mode. In the manual mode, the user can change manually of the two functions (soft tissue/structure targets or tough tissue/structure targets). In the automatic mode, the user need not change any system functions, the system detects the tissue type based on the torque or other detected operating characteristic to determine proper cutting mode.

In other operational variations, the cutting head may remain in the “closed” state this is the base state. When this state is desired, the user need only press a button, and the head will rotate into the closed state. Optionally, the insertion or removal process can be only in the closed state (to avoid injuries). In some modes of operation, there is an interaction with a footswitch button during the operation, the inner cutting head stops in a different position—usually in an “open” (“free”) state. In the closed state, the fluid circulation (flushing) goes through on the flush (bypass) channel, which is a hole or slot on the fixed outer head's backface.

In yet another alternative embodiment, used alone or in combination with the electronic means of closing the head, there is also a mechanical means of rotating the head back to closed state. In one aspect, a mechanical head closure implementation could be, for example, achieved by a resector head geometry that urges, bias, or forces the resector head to close when the cutting head is being pulled into the scope or other access device that the resector or cutting tool is deployed through.

In other operational variations of a soft tissue function, the curved inner cutting head rotates in an oscillated pattern. One exemplary pattern is 3 times clockwise (CW) and then three times counter clockwise (CCW). Additionally, it will be appreciated that with each change of rotation a different main cutting edge of the outer head (i.e., the straight cutting edge) will be engaged when the rotating direction changes. In the tough tissue function, direction of rotation does not vary so the engaged cutting edges do not change. Optionally, a user may manually selected the tough tissue function to be performed in the opposite direction of rotation.

As best seen in the view of FIG. 4D, the excentric axis feature is needed for better “milling” (tough tissue cutting) performance. The smooth back face of the cutting element comes from the excentric design. Advantageously, the excentric device feature is so multifunctional, but may be used it in the first case for the hysteroscopy tough/calcified—and soft as well—uterus tissue resection. In still further possible operating modes, the cutting head may be used in other types of operations including those with a bone-shaver (e.g., orthopedic), soft tissue resector, or both of them together, selected on demand based on clinical circumstances.

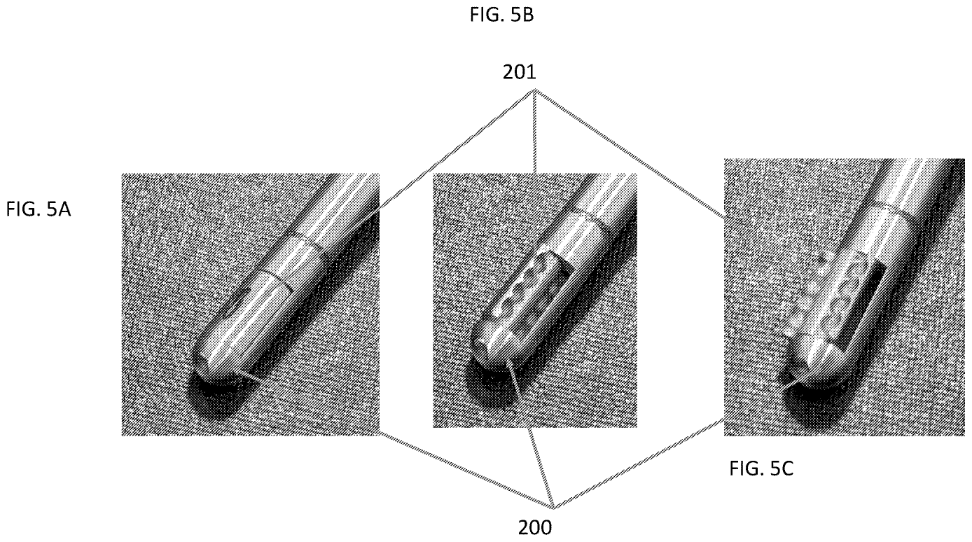

FIGS. 5A-5C illustrate a perspective view of a prototype of an embodiment of a cutting head. FIG. 5A illustrates the cutting head at the distal end of a device in a closed configuration in which no cutting surface is exteriorly presented. FIG. 5B illustrates the cutting head of FIG. 5A with the inner cutting head half opened exposing one scalloped cutting edge and one straight edge of the outer head. FIG. 5C is a view of the cutting head FIG. 5A with the inner cutting head fully open exposing both scalloped cutting edges and both straight cutting edges of the outer head (only one visible in this view).

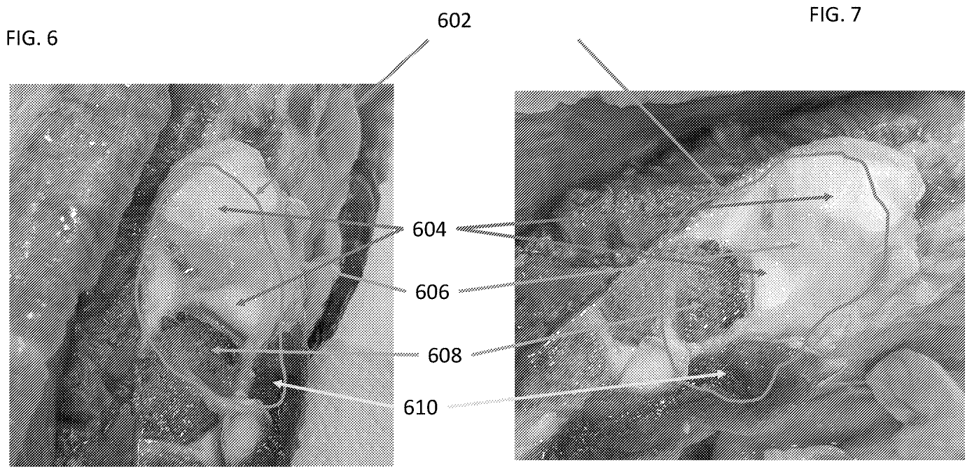

FIG. 6 is a first perspective view of a resected portion 602 of a cadaver limb showing the effective removal of cartilage 604, ligament 606, bone 608 and soft tissue 610 via operation of the prototype cutting head of FIGS. 5A-5C.

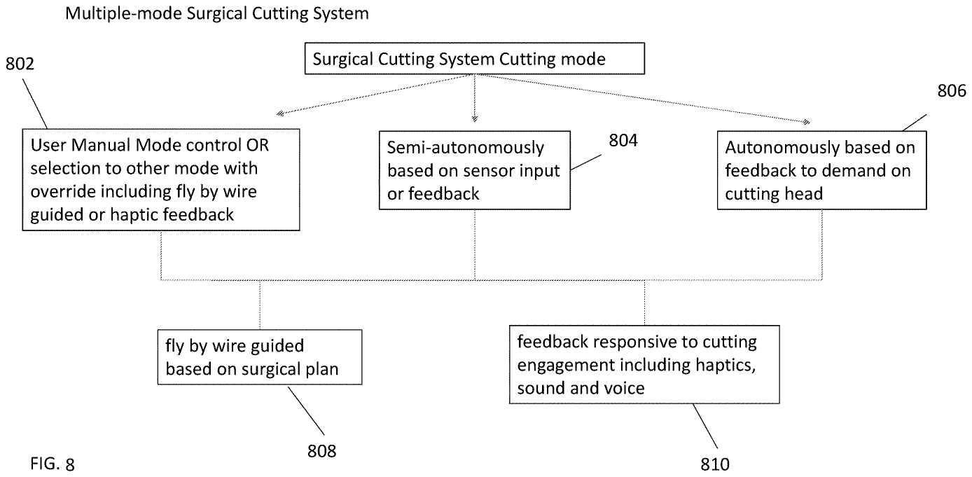

FIG. 7 is a second perspective view of the resected portion of a cadaver limb of FIG. 6 showing the effective removal of cartilage, ligament, bone and soft tissue via operation of the prototype cutting head of FIGS. 5A-5C. FIGS. 6 and 7 are an exemplary demonstration of how the multi-mode cutting head is suited for the cutting of a variety of soft, hard and tough or calified tissue unlike conventional surgical cutters that are optimized or configured for only one cutting modality. In embodiments of the inventive cutting head, a different cutting mode may be selected and switched between readily using the user controls or semi- or autonomous mode operation. Optionally, the user may also manually set the RPM within a pre-selected operating range, or the oscillating mode of the rotating head.

FIG. 8 is a flow chart detailing a number of different operating modes for the cutting head surgical system. It is to be appreciated that in some embodiments, the cutting surgical system is adapted and configured to sense what type of tissue or structure a cutting surface is in contact with or engaged with either initially before cutting or intermittently during a cutting operation. In still further aspects, the cutting parameters may be adjusted for different operational modes including, without limitation, 1) user demand 802 or 2) semi-autonomously 804 or 3) autonomously 806 responding to the demands sensed by the system.

User manual mode control can allow selection to another mode with override including fly by wire guided or haptic feedback. Semi-autonomous mode can comprise using sensor input or feedback for operation. Autonomous mode can be based on feedback to demand on the cutting head.

The number and types of cutting performance are also available to a user via a suitable user interface or control system interface at the proximal end of the device within the handle or part of a control interface. Additionally or optionally, the system may be used in a test and detection mode 808 in which various portions of the surgical site are engaged and a tissue engagement index is consulted to aid the user in an appropriate cutting mode for each area tested as an aid to surgical planning. In one aspect, the use of this feature would improve safety by ensuring that the lowest power cutting mode is selected for most effective removal of targeted tissue while retaining safety to surrounding tissue which may be harmed if an aggressive cutting mode is used in an easier to cut tissue.

In an exemplary cutting mode 810, the user can receive feedback responsive to cutting engagement, including haptics, sound, and voice feedback.

In one exemplary cutting mode control, the user can increase tissue removal performance through the user interface of the device, by changing settings.

In another exemplary cutting mode control, the system can autonomously adjust some parameters, while other remain under direct user control. For example, the system could drop rpm when sensing lower resistance. This would be a safety feature, for example. Another example is stepping up rpm when sensing higher resistance. This is an improved performance measure.

In yet another exemplary cutting mode control embodiment, the system can autonomously take control over all performance parameters. An example would be automatic shut off, also a safety feature.

In still other exemplary cutting mode control embodiments, the parameters that can be adjusted such a way are rpm, direction of rotation, different forms of alternating direction, including vacuum operation and suction level applied, as well as flow of fluids into the surgical site. In still another aspect, a cutting rate sensor or other measurement sensor, such as ultrasound measuring distance, or accelerometer measuring movement, may be used to provide inputs to the system. These or other sensor inputs could change with different sampling rates as well as combinations and considerations of different inputs or performance parameters of the system depending upon the surgical scenario, surgical preference setting or overall safety settings to increase a margin of safety for system operation.

Advantageously, these and other parameters that could be sensed by the system in order to adjust performance would include measures of tissue resistance detected at the level of the motor or by directly measuring it by ultrasound or the optical system, heat generation, detection of motion parameters, detection of tissue removal rate, and other such sensors used to determine parameters related to the surgical field, the tissue or tool operation. Moreover, one overall benefit of autonomous operation is increased safety coupled with reduced length of procedure. Also, the surgeon will not have to use multiple tools, as this one can handle all tissue densities and levels of calcification. This further reduces time (no swaps), cost (one device as opposed to multiple) and safety (one device to learn, not many).

In one aspect, advantageously, the cutting head is actually functioning as a hybrid head such as one having capabilities of a resector and a bone shaver together. For example, if a user wants to cut soft tissue and ligament, the scalloped inner head rotates around (360 deg) typically 3× back (CW) and 3× forth (CCW) on RPM 2000-4000 providing operational characteristics much like a resector. Still further, if a user wants to cut cartilage and bone, the scalloped inner head rotates in only one direction—e.g., CW—on RPM 4000-8000 providing operational characteristics much like a shaver or a milling tool. The outer head with straight cutting edges is fixed all the time. The straight cutting edges are more robust than the scalloped, but the scalloped shape on the inner head also provides the functionality for clawing into the targeted tissue. The secondary cutting edges need to cut the tissue, which is stuck into the vacuum channel inlet (See, e.g., FIGS. 4A-4D). The scalloped inner head's rotating axis and the outer head's central axis are excentered. The resecting line is out of the shape of the outer head, but the head is closeable when it needs to go through on a seal, for example. The backface opening (slot) on the outer head allows a bypass channel for fluid circulation when the head is closed.

In one specific implementation of the inventive surgical cutter, the same design is used for soft tissue, ligament, cartilage, and bone resecting. Diameter can be in a closed position of 3-8 mm. Current OD 5.33 mm. The cutting window can be 3-15 mm. One prototype design includes a 8.2 mm cutting window. The resecting line is out of the closed head OD in the radial direction. RPM with oscillation for soft tissue and ligament 2000-4000 rpm. RPM with no oscillation for cartilage and bone 4000-8000 rpm. The cutting edges can be in different shapes, waves, and angles (scissors effect, different profile angles of the cutting edges, various numbers of nips on the inner head, nips on the outer head, and various numbers of nips on the outer head). There may also be provided bypass slots on the heads in various numbers and sizes. Additionally or optionally, the vacuum channel may be variously sized and shaped.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

By way of an additional example, the surgical head described herein would be included in as part of a system with resecting motor, fluid system (with pump), specimen trap, controller, and hysteroscope. In additional aspects, there may also be a mixture of reusable parts and disposable parts in the system. In one exemplary embodiment, there reusable components of the system, may include, resecting motor, pump (without fluid lines), controller and an access scope such as a hysteroscope. The disposable parts, may for example, include the resector head, the fluid lines and the specimen trap. Alternative divisions between disposable and reusable are possible.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A surgical cutting head, comprising:
a proximal end having a handle,
a distal end having a cutting head with an outer cutting head having an opening and two straight cutting edges and an inner cutting head positioned for eccentric rotation relative to the opening and in cutting relation to the two straight cutting edges.

2. The surgical cutting head of claim 1, further comprising:
a motor control system adapted and configured to control the direction and speed of rotation of the inner cutting head.

3. The surgical cutting head of claim 2, wherein the motor control system may be operated in a manual mode, a semiautonomous mode or an autonomous mode.

4. The surgical cutting head of claim 1, wherein the cutting head has a closed mode where no cutting surface is exposed or only a smooth exterior surface of the cutting head is presented.

5. The surgical cutting head of claim 4, further comprising a push button, a selector switch, a footswitch or a voice activated control system adapted and configured to automatically transition the cutting head into the closed mode.

6. The surgical cutting head of claim 3, further comprising: one or more sensors coupled to a portion of the cutting head and a computer implemented tissue characterization and mode selection tissue cutting control system in electronic communication with the one or more sensors wherein based at least in part on a signal from the one or more sensors the computer implemented cutting control system recommends or automatically adjusts the motor control system between the manual mode, the semi-autonomous mode and the autonomous mode.

7. The surgical cutting head of claim 6, wherein the computer implemented tissue characterization and mode selection tissue cutting control system is configured to automatically transition the cutting head to a closed mode where no cutting surface is exposed based on a determination of signals received from the one or more sensors.

8. A method of removing bone, ligament, muscle, soft tissue, calcified tissue, scarred tissue, tumor or other targeted tissue using the device of claim 6.

9. A method of removing bone, ligament, muscle, soft tissue, calcified tissue, scarred tissue, tumor, or other targeted issue, comprising
advancing a device comprising a cutting head with an outer cutting head having an opening and two straight cutting edges and an inner cutting head positioned for eccentric rotation relative to the opening and in cutting relation to the two straight cutting edges to a target tissue;
selecting one or more cutting parameters based on properties of the target tissue; and
rotating the inner cutting head relative to the outer cutting head, thereby removing tissue from the target tissue.

10. The method of claim 9, further comprising selecting a mode of operation of the cutting device.

11. The method of claim 10, wherein selecting a mode of operation comprises selecting between manual control, semi-autonomous control or autonomous control.

12. The method of claim 9, further comprising engaging various portions of a target tissue site with the cutting head to aid in selection of an appropriate cutting mode for the various portions of the target tissue site.

13. The method of claim 9, further comprising modifying the mode of operation or mode of cutting based on feedback from sensors on the cutting head.

14. The method of claim 9, wherein rotating the inner cutting head relative to the outer cutting head comprises rotating the inner cutting head back and forth about 2-5 times at an RPM of about 2000-4000 RPM.

15. The method of claim 14, further comprising cutting through soft tissue.

16. The method of claim 9, wherein rotating the inner cutting head relative to the outer cutting head comprises rotating the inner cutting in one direction at an RPM of about 4000-8000 RPM.

17. The method of claim 15, further comprising cutting through cartilage and/or bone.
